(19) United States
(12) Patent Application Publication
(10) Pub. No.: US 2005/0245847 A1
(43) Pub. Date: Nov. 3, 2005

(54) Wire guide apparatus

(75) Inventor: Darin G. Schaeffer, Bloomington, IN (US)

Correspondence Address: BRINKS HOFER GILSON & LIONE/CHICAGO/COOK, PO BOX 10395, CHICAGO, IL, 60610, US

(73) Assignee: Cook, Inc.

(21) Appl. No.: 11/118,635

(22) Filed: Apr. 29, 2005

Related U.S. Application Data
(60) Provisional application No. 60/567,222, filed on Apr. 30, 2004.

(51) Int. Cl. A61B 5/00

(52) U.S. Cl. 600/585

(57) ABSTRACT

An apparatus is provided for introducing a wire guide into a vessel through a medical device. The apparatus generally includes a body, a distal tip, and a first advancement wheel. The body defines a first axial passageway therein. The distal tip of the body defines a male Luer fitting and terminates in a taper having an angle relative to an outer surface of the male Luer fitting. The advancement wheel is rotatably attached to the body and positioned adjacent the first axial passageway. The first wheel defines a grasping area contacting the wire guide and axially translates the wire guide relative to the body. A holder is preferably attached to a proximal end of the body and stores a portion of the guide wire for delivery into the vessel.

### CROSS-REFERENCE TO RELATED PATENT APPLICATIONS

This patent application claims the benefit of U.S. provisional patent application Ser. No. 60/567,222, filed on Apr. 30, 2004.

### FIELD OF THE INVENTION

The present invention relates generally to wire guides for interventional medical procedures, and more particularly relates to a device for introducing a wire guide into a vessel.

### BACKGROUND OF THE INVENTION

A wire guide is a device used in interventional procedures to facilitate the placement of other devices such as catheters, stents, valves, filters, etc., within the vessels of the body. An exemplary wire guide is disclosed in U.S. Pat. No. 3,547,103, and generally includes a coil spring circumscribing a mandrel to provide a wire guide having sufficient rigidity and flexibility for passage through the vascular system. In many designs, such wire guides includes a distal end defining a J-shape or other curved shape. This distal end shape is designed to reduce the potential for trauma caused to the vessels.

In these interventional procedures, an introducer needle is used to gain access to a vessel and place the wire guide within the vascular system. To pass the wire guide through the introducer needle, the J-shaped atraumatic distal tip must first be straightened for insertion into the needle. Once the wire guide is positioned within the needle, the needle is inserted into a vessel, and then the wire guide is advanced to the desired location within the vascular system. The distal tip of the wire guide resumes its curved shape upon exiting the needle for safe passage through the vascular system. With the distal tip of the wire guide positioned at the predetermined location, a device such as a catheter may be advanced over the wire guide in order to position the catheter at the predetermined location within the vascular system.

Unfortunately, introducing a wire guide in this manner requires numerous steps and a complicated, lengthy procedure. In particular, the wire guide must first be straightened by pulling the wire guide through a straightener, and then introduced into the needle through the straightener. Then the wire guide is hand fed to the predetermined location. The straightener is then typically removed, and the wire guide must be held in place relative to the introducer needle during removal.

Accordingly, there exists a need to provide a device which allows an individual medical professional to straighten a wire guide, position it within an introducer needle, introduce the wire guide into the vessel, and advance the wire guide to a desired location within the vascular system in a simple and efficient manner.

### BRIEF SUMMARY OF THE INVENTION

One embodiment of an apparatus for introducing a wire guide into a vessel through a medical device is provided in accordance with the teachings of one embodiment of the present invention. The apparatus generally includes a body defining a first axial passageway therein. A distal tip of the body defines a male Luer fitting and terminates in a taper having an angle relative to an outer surface of the male Luer fitting. A first advancement wheel is rotatably attached to the body and positioned adjacent the first axial passageway. The first advancement wheel defines a grasping area contacting the wire guide and axially translating the wire guide relative to the body. A valve may be positioned within the body and distal to the first advancement wheel. The valve is structured to sealingly engage the wire guide.

According to more detailed aspects, the distal tip is angled at an increased slope in the distal direction. Preferably, the distal tip includes a compound angle defined by a first conical portion and a second conical portion, the first conical portion having a slope greater than the second conical portion. The second conical portion forms a portion of the male Luer fitting. The apparatus may further include a rotatable nut positioned on the exterior of the distal tip that works in conjunction with the second conical portion to define the male Luer fitting. The apparatus is configured for one-handed operation wherein a wire guide may be straightened and introduced and traversed through the vessel via one-handed operation of the apparatus.

According to still further detailed aspects, a port defines a second passageway in communication with the first passageway. The port is located distally relative to the valve. The distal tip preferably defines a straightener suitable to straighten a curved distal tip of the guide wire. The apparatus may also include a holder connected to a proximal end of the body and in communication with the first axial passageway. The holder stores a portion of the wire guide for delivery into the vessel, and is preferably a spiral holder. A lock selectively engages the advancement wheel. When the first advancement wheel is radially adjustable, the lock selectively and independently fixes both the rotation and the radial position of the first advancement wheel.

Another embodiment of the apparatus generally includes a body, a straightener, a first advancement wheel, a valve and a flushing port. The body defines a first axial passageway therein. The straightener is attached to a distal end of the body and defines a second axial passageway in communication with the first axial passageway. The advancement wheel is rotatably attached to the body and positioned adjacent the first axial passageway. The wheel defines a grasping area contacting the wire guide and axially translating the wire guide relative to the body. The valve is located distally relative to the pair of advancement wheels and sealingly engages the wire guide to prevent fluids from entering the body and reaching the advancement wheel. The port is located distally from the valve and defines a second axial passageway in communication with the first and/or second passageways.

According to more detailed aspects, the straightener may be unitarily formed with the body. Likewise, the port may be unitarily formed with the body and straightener. A holder may be connected to a proximal end of the body and is in communication with the first axial passageway. The holder stores a portion of the guide wire for delivery into the vessel, and is preferably a spiral holder.

### BRIEF DESCRIPTION OF THE DRAWINGS

The accompanying drawings incorporated in and forming a part of the specification illustrate several aspects of the present invention, and together with the description serve to explain the principles of the invention. In the drawings:

FIG. 1 is a plan view of a wire guide kit having a wire guide apparatus constructed in accordance with the teachings of the present invention;

FIG. 2 is perspective view of the wire guide apparatus depicted in FIG. 1;

FIG. 3 is a side view of the wire guide apparatus shown in FIGS. 1 and 2; and

FIG. 4 is a side view similar to the FIG. 3 but showing an alternate embodiment of the wire guide apparatus constructed in accordance with the teachings of the present invention;

FIG. 5 is an enlarged, partially cut-away, side view of a lock forming a portion of the wire guide apparatus depicted in FIG. 4; and

FIG. 6 is an enlarged, partially cut-away, cross-sectional view of another alternate embodiment of the wire guide apparatus constructed in accordance with the teachings of the present invention.

### DETAILED DESCRIPTION OF THE INVENTION

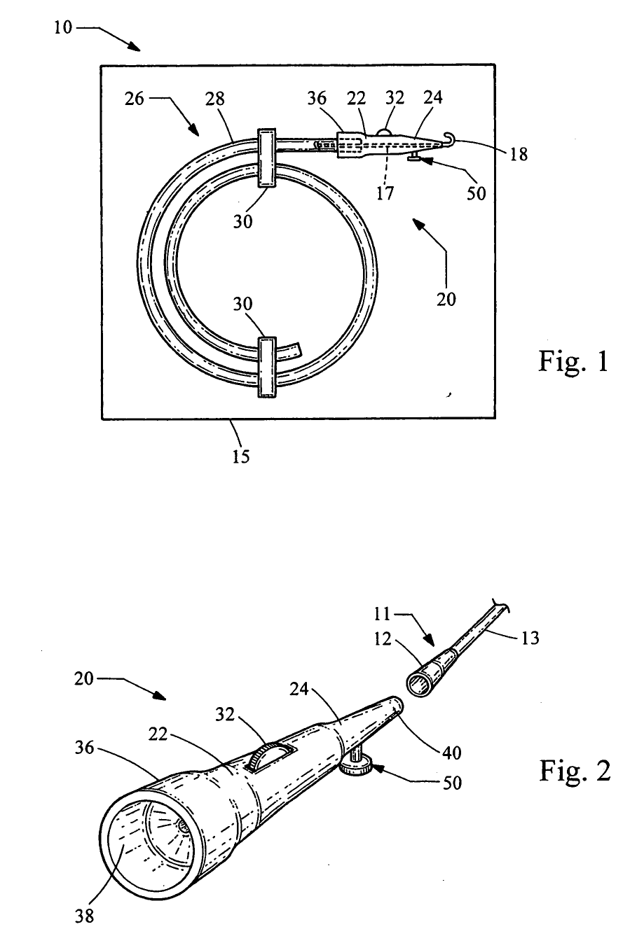

Turning now to the figures, FIG. 1 depicts a plan view of a wire guide kit 10 having a wire guide apparatus 20 constructed in accordance with the teachings of the present invention. The kit 10 preferably includes sealed packaging 15 which can be used to maintain the wire guide apparatus 20 and the wire guide 17 contained therein in a suitable condition for use, and preferably in a sterile condition.

As best seen in FIGS. 1 and 2, the wire guide apparatus 20 generally includes a body 22, a straightener 24, and a holder 26. The holder 26 is preferably a spiral holder having a flexible tubular member 28 wound into a spiral configuration and held in place by clips 30, as is shown in FIG. 1. The body 22 includes a proximal end 36 which defines an opening 38 therein. The opening 38 is sized to receive a free end of the holder 26, and in particular a free end of the flexible tube 28. The outer diameter of the tube 28 and the inner diameter of the proximal end 36 defining the opening 38 are sized to provide a friction fit between the body 22 and the holder 26. In this way, the spiral holder 26 is selectively attachable to the body portion 22.

As will be described in further detail herein, the body 22 has an advancement wheel 32 positioned therein for advancing the wire guide 17 through the apparatus 20. A straightener 24 is attached to a distal end of the body 22. The straightener 24 tapers to a narrow distal end 40. The distal end 40 is sized and structured for connection to a medical device 11, for example a needle or catheter. As is known in the art, the medical device 11 shown in the drawings is an introducer needle and generally includes a hub portion 12 and a needle portion 13. The opening defined in the hub 12 receives the tapered distal end 40 of the straightener 24, and forms a friction fit therebetween that allows the wire guide apparatus 20 to be selectively attached to the medical device 11. It will be recognized that the apparatus 20 may also be used with other devices, such as a check-flo valve or catheter, as will be described later herein.

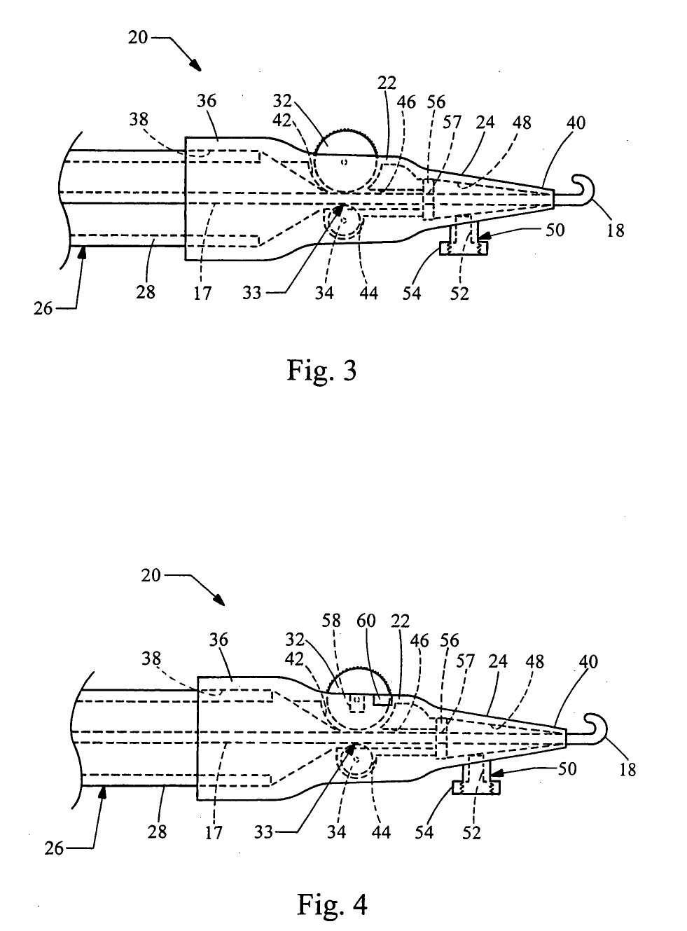

Further details of the wire guide apparatus 20 will now be given with reference to FIG. 3. The body portion 22 defines a slot 42 therein, the slot 42 sized to receive the first advancement wheel 32. A second advancement wheel 34 is also provided, and the body 22 defines a second slot 44 which is sized to receive the second advancement wheel 34. The first and second advancement wheels 32, 34 are rotatably attached to the body 22, preferably by way of a pin or axle. The first and second wheels 32, 34 are positioned relative to each other to define a grasping area 33 therebetween. As shown in the figure, the body 22 further defines a first axial passage 46 extending therethrough that is sized to receive the wire guide 17. The first and second advancement wheels 32, 34 are in communication with the first passageway 46, and more particularly the grasping portion 33 engages the wire guide 17 which is positioned within the first passageway 46.

The first advancement wheel 32 is preferably positioned only partially within the body 22, such that a portion of the advancement wheel 32 protrudes from the body 22 and is accessible to a user of the apparatus 20 for advancing the wire guide 17. The second advancement wheel 34 is preferably smaller than the first wheel 32 and is contained within the body 22. It will be recognized that the single advancement wheel 32 could be used without the opposing second wheel 34. More specifically, the body 22 could define a flat surface in place of the second wheel 34 to thereby still define a gripping area 33, wherein the first wheel 32 has a tacky outer surface that effectively grips the wire guide 17 and advances the wire guide 17 relative to the body 22.

The straightener 24 tapers radially inwardly as the straightener 24 extends towards the distal end 40. The straightener 24 defines a second axial passageway 48 therein. The second passageway 48 has been shown as also being tapered or conical, corresponding to the tapered shape of the straightener 24. It will be recognized that the second passageway 48 could be shaped differently, such as cylindrical in shape. The portion of the second passageway 48 proximate the distal end 40 is of a relatively narrow size but still sufficiently large to receive the wire guide 17. When the second passageway 48 is not tapered, the passageway is sized to receive the wire guide 17 and is preferably sized to correspond with the first passageway 46, and to keep the distal end of the wire guide 17 straight. The second passageway 48 is in communication with the first passageway 46. Preferably, the straightener 24 is integrally formed with the body 22 and thus the first and second passageways 46, 48 may also be integral. The first passageway 46 enlarges radially and is in communication with the opening 38 defined by the proximal connection end 36 of the body 22. Thus, the wire guide 17 can extend through the second passageway 48 of the straightener 24, into and through the first passageway 46 of the body 22, and into the tubular member 28 of the spiral holder 26, as shown in FIG. 3.

As shown in FIG. 3, the wire guide apparatus 20 preferably includes a valve 56 situated therein. As shown, the valve 56 includes an aperture 57 defined therein which is sized to receive and sealingly engage the wire guide 17. The outer periphery of the valve 56 is fluidically sealed against the body 22 and/or the straightener 24. The valve 56 may be positioned at the juncture of the first passageway 46 and the second passageway 48. However, the valve 56 may be positioned at any point along the first and second passageways 46, 48, so long as the valve 56 is positioned upstream or distally relative to the advancement wheels 32, 34. In this way, any fluid entering the apparatus 20 by way of the straightener 24 and its second passage 48, or the port 50 and its passageway 52, is prevented from flowing into the slots 42, 44 which receive the advancement wheels 32, 34 and the holder 26.

A port 50 is attached to the straightener 24, and defines a third passageway 52 therein. The port 50 preferably includes a fitting 54 such as Luer fitting which allows the port 50 to be used as a flushing port, or for delivery of other materials by injection. The third passageway 52 of the port 50 is in communication with second passageway 48, and is positioned distally from the valve 56. Stated another way, the port 50 is positioned between the valve 56 and the free distal end 40 of the straightener 24. It will be recognized that the port 50 could be supplemented or replaced by using the proximal end 36 of the body 22 as a port, either through the tubular holder 26 or without the holder 26. In the latter case, the proximal end 36 could be shaped as a Luer fitting. In such an embodiment, the valve 56 will be structured as a one-way valve permitting fluids to pass distally while preventing fluids from flowing proximally. Such one-way valve configurations are well known in the art and can readily be used in the apparatus 20.

In operation, the wire guide apparatus 20 is prepackaged as a kit 10 within a wrapper 15, and includes a wire guide 17 positioned therein with the J-shaped distal end 18 of the wire guide 17 protruding from the straightener 24. Alternatively, when the wire guide apparatus 20 is provided as a separate device, a proximal free end of the wire guide 17 may be fed into the apparatus 20 through the second passageway 48 formed in the distal end 40 of the straightener 24. Once the wire guide 17 has been manually fed through the second passageway, the valve 56 and a portion of the first passageway 46, the wire guide 17 reaches the grasping area 33 formed between the advancement wheels 32, 34. At this point, the first protruding advancement wheel 32 may be rotated (clockwise or to the right in FIG. 3) in order to advance the wire guide 17 relative to the apparatus 20 and its body 22. The wire guide 17 will freely enter the holder 26 and follow the coiled path formed by the flexible tube 28.

Once the wire guide 17 has been positioned within the apparatus 20, the first advancement wheel 32 may be rotated such that the wire guide 17, and in particular the J-shaped distal end 18 moves inside of the second passageway 48 and within the straightener 24. The narrow distal tip 40 will force the J-shaped end 18 to straighten. In this condition, the apparatus 20 and in particular the distal tip 40 of the straightener 24 may be attached to the hub portion 12 of the medical device 11 (FIG. 2). Once attached, the first advancement wheel 32 may then be rotated in the opposite direction (counterclockwise or to the left in FIG. 3) in order to advance the wire guide 17, and in particular the straightened distal end 18 into the introducer 11 and its needle portion 13. As is known, once the wire guide 17 exits the needle portion 13 within the vessel, the distal end 18 will resume its J-shape such that the wire guide 17 is atraumatic to the vessel.

Alternatively, vessel access may already be provided by a catheter or the like, and the distal end 40 of the straightener 24 may be fitted to a proximal end of the catheter for feeding the wire guide 17 into the vessel through the catheter. In this case, it will be seen that by connecting the apparatus 20 and its distal end 40 to the catheter, the wire guide 17 may be translated relative to the catheter, while at the same time the position of the catheter can be maintained by limiting the movement of the apparatus 20.

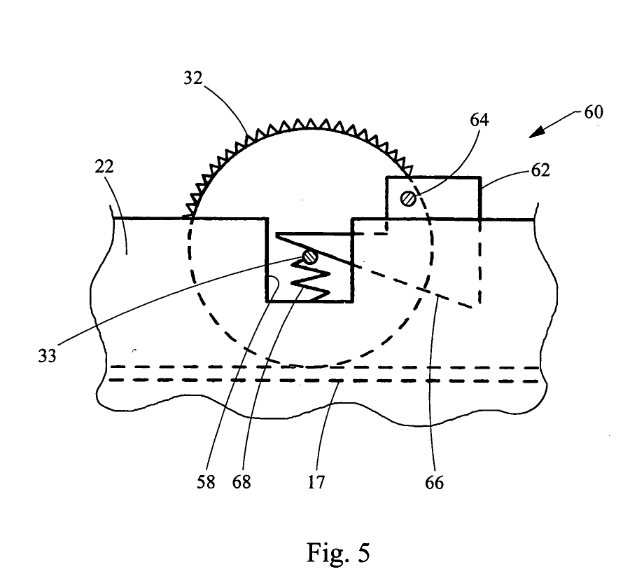

Once the distal end 18 of the wire guide 17 is positioned within the vessel, the advancement wheels 32, 34 may continue to be used in order to advance the wire guide 17 through the vascular system. In this manner, excellent control over the wire guide 17 is permitted throughout the procedure of placing the wire guide 17. It will be recognized by those skilled in the art that the design of the apparatus 20 permits the one-handed operation of the apparatus to deliver and maneuver the wire guide 17 through the vascular system. More specifically, only a single-hand is needed to grasp the apparatus 20 from the kit 10, and retract the wire guide 17 to straighten the distal end 18 thereof. Then, once the apparatus 20 is connected to either an introductory needle, catheter, or other device providing vessel access, the apparatus 20 and its advancement wheel 32 may be operated to translate the wire guide through the vasculature, and relative to the introducer 11, catheter or other access device. Likewise, and as described further below with reference to FIGS. 4 and 5, when the apparatus 20 includes a lock 60, the proximity of the lock 60 relative to the advancement wheel 32 permits single-handed operation of the lock 60. The lock can both engage/disengage the wire guide 17, as well as fix the position of the advancement wheel 32 (relative to the body 22), thereby providing increased flexibility and control over the wire guide 17 while at the same time providing simple one-handed operation.

If it is desired to remove the medical device 11, the advancement wheel 32 may be rotated (or the apparatus 20 may be pulled) while holding the wire guide 17 in place in order to remove all of the wire guide 17 from the apparatus 20 and its holder 26. Then, the medical device 11 may be removed. If desired, the proximal free end of the wire guide 17 may then be repositioned within the apparatus 20 and the excess wire translated into the holder 26 such that the advancement wheels 32, 34 may continue to be used to traverse the wire guide 17 through the vascular system.

At any point in the process, it will also be recognized that the port 50 may be used to inject a substance through the passageway 52, the second passageway 48 and through the medical device (for example a needle or catheter) into the vessel. A flushing of the vessel is often desired and thus can be performed with the apparatus 20 of the present invention. Further, the moving parts of the apparatus 20, and namely the advancement wheels 32, 34, are protected from any bodily fluids or other materials which might contaminate or impair the operation of the wheels 32, 34.

An alternate embodiment of the wire guide apparatus 20 has been depicted in FIGS. 4 and 5. This embodiment is substantially identical to the embodiment depicted in FIGS. 1-3, and common reference numerals have been used. In this embodiment the apparatus 20, and in particular the main body 22, has been formed with a slot 58 which permits the first advancement wheel 32 to move radially relative to the first passageway 46 and the central axis of the apparatus 20. The slot may provide distinct radial positions for the wheel 32 through tabs, grooves or friction fittings. As such, the first advancement wheel 32 is selectively positionable relative to the wire guide 17.

A lock 60 has also been shown positioned proximate the advancement wheel 32 and attached to the body 22. The lock 60 may be selectively activated by the user of the apparatus 20 in order to fix the position of the wheel 32. In this way, the lock 60 may be used to prevent rotation of the advancement wheel 32, and hence control the position of the wire guide 17. Further, the lock 60 may be used to fix the radial position of the advancement wheel 32, i.e. its position within the slot 58. Thus, the advancement wheel 32 may be moved radially to selectively engage or disengage the wire guide 17. This is helpful not only in advancing the wire guide 17 through the vascular system, but also allows for easy introduction and removal of the wire 17 from within the apparatus 20.

An enlarged, partially cut-away view of the lock 60 has been depicted in FIG. 5. The lock 60 generally includes a cam member 62 connected to the body 22 in a manner permitting axial translation, such as by a slot formed in the body (not shown). The cam member 62 includes an adjustable locking pin 64 for selectively engaging and fixing the first advancement wheel 32. The cam member 62 also includes an angled cam surface 66 which contacts the axle 33 of the first advancement wheel 32. The axle 33 and wheel 32 are biased upwardly by a spring or other resilient member 68. It will be seen that as the cam member 62 is moved distally (to the left in FIG. 5), the cam surface 66 forces the axle 33 and advancement wheel 32 downwardly in the slot 58, thereby permitting the selective engagement of wire guide 17. It will be recognized by those skilled in the art that numerous locking and latching mechanisms can be employed to fix or restrict the rotation of the first advancement wheel 32 as well as fix or restrict the radial position of the wheel 32.

Accordingly, the present invention provides an apparatus that permits an individual to easily and quickly straighten the distal tip of a wire guide, position the wire guide within an introducer needle, and place the wire guide within a vessel. Further, the apparatus facilitates the advancement of the wire guide through the vascular system by way of adjustable advancement wheels. A valve ensures proper operation of the device and its advancement wheels, while a port is provided for flushing or injecting additional substances. The apparatus and wire guide are preferably provided as a pre-packaged kit for expedient preparation and ease of use.

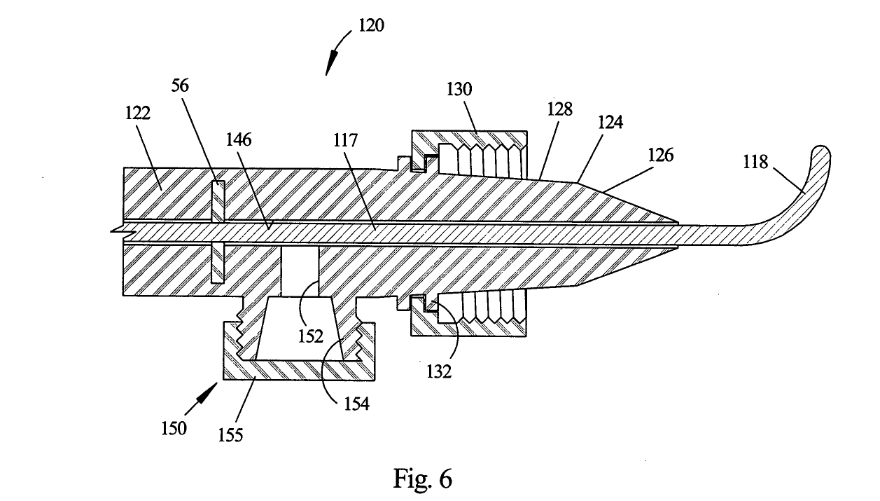

FIG. 6 depicts yet another embodiment of the wire guide apparatus 120 constructed in accordance with the teachings of the present invention. The figure depicts the distal end of the apparatus 120, as all other parts are the same as the previous embodiments. In this embodiment, the main body 122 includes a distal tip 124 that defines a male Luer fitting and terminates in a linear or non-linear taper having an angle relative to an outer surface of the male Luer fitting. In the embodiment of FIG. 6, the distal tip 124 includes a first conical portion 126, positioned distally from a second conical portion 128, and a compound angle at the intersection of the two. The first conical portion 126 tapers at an angle that is greater than the angle of the second conical portion 128 (i.e. relative to the guide wire 117). It will be recognized by those skilled in the art that the first conical portion 126 may take other forms, such as curved outer surfaces or a single curvature of increasing slope, to achieve the same result as described below.

Generally, this compound angled distal tip section 124 adapts the apparatus 120 for use with both the hub 12 of the medical device 11 (FIG. 2) as well as for use with various catheters including those having a Luer fitting. More specifically, the larger taper of the first conical section 126 permits the extreme distal tip (i.e. where the wire guide 117 protrudes from the apparatus 120) to be positioned as closely as possible to the needle portion 13 inside of the hub 12 of the medical device 11. This minimizes the possibility of the distal tip 118 of the wire guide 117 resuming its curvature before it enters the needle portion 13, thereby improving reliable passage of the wire guide 117.

Additionally, the second portion 128 of the distal tip 124 not only allows the apparatus 120 to be used with hubs or connecting pieces having different internal slopes, but also facilitates the interconnection of the apparatus 120 with catheters or other devices having a Luer fitting. That is, the second conical portion 128 is sloped at an angle corresponding to those of male Luer fittings, which in combination with a captive but rotatable nut or collar 130, defines a lockable Luer connector. Alternatively, the collar 130 may be fixed in position. For example, the second portion 128 may be configured as a male Luer fitting as specified by International Standard Reference Number ISO 594/1-1986(E) “Conical fittings with a 6% (Luer) taper for syringes, needles and certain other medical equipment—Part 1: General requirements”, the contents of which are hereby incorporated by reference as if fully set forth herein. The collar 130 may be configured with internal threads as specified by International Standard Reference Number ISO 594-2:1998(E) entitled “Conical fittings with a 6% (Luer) taper for syringes, needles and certain other medical equipment—Part 2: Lock fittings”, the contents of which are hereby incorporated by reference as if fully set forth herein. A pair of flanges 132 capture the collar 130 on the exterior of the apparatus 120, although a single distal flange 132 could be employed.

For example, the flushing port 150 of the present embodiment has been shown as including the female fitting 154 of a Luer connector which defines an internal passageway 152 therein. The passageway 152 permits fluids to pass into the main passageway 146 defined by the main body 122, which also forms the internal passageway of the distal tip section 124. When not in use, the flushing port 150 may be closed off by way of a cap 156 as shown in the figure. The cap 156 could also be replaced with a relief valve in order to prevent fluids from traversing into the wheels in the unlikely event the valve 156 is unable to withstand the fluid pressure within the passageway 146.

It will also be seen that the distal tip 124 and Luer fitting 130, as well as the flushing port 150, are located distally from the valve 156 which typically comprises a rubber gasket such as an O-ring. The O-ring is held in place by a pair of bosses 157 although other retaining structures may be readily employed as is known in the art. In this manner, various catheters, needles, syringes and other similar devices may be attached to the wire guide apparatus 120 and fluidly connected to the internal passageway 146 without the need for adapting the particular device to seal against the wire guide 117.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A wire guide apparatus for introducing a wire guide into a vessel through a medical device, the apparatus comprising:
a body defining a first axial passageway therein;
a distal tip of the body defining a male Luer fitting and terminating in a taper having an angle relative to an outer surface of the male Luer fitting;
a first advancement wheel rotatably attached to the body and positioned adjacent the first axial passageway, the first advancement wheel defining a grasping area contacting the wire guide and axially translating the wire guide relative to the body.

2. The apparatus of claim 1, comprising a valve positioned within the body and distal to the first advancement wheel, the valve structured to sealingly engage the wire guide.

3. The apparatus of claim 1, wherein the distal tip includes a compound angle defined by a first conical portion and a second conical portion, the first conical portion having a slope greater than the second conical portion.

4. The apparatus of claim 3, wherein the second conical portion forms a portion of the male Luer fitting.

5. The apparatus of claim 1, further comprising a collar positioned on the exterior of the distal tip.

6. The apparatus of claim 1, wherein the apparatus is configured for one-handed operation, and wherein a wire guide may be straightened and introduced and traversed through the vessel via one-handed operation of the apparatus.

7. The apparatus of claim 4, wherein the valve is a one-way valve structured to permit fluid to flow distally past the valve.

8. The apparatus of claim 1, further comprising a port defining a second passageway, the second passageway in communication with the first passageway.

9. The apparatus of claim 8, wherein the port is located distally relative to the valve.

10. The apparatus of claim 1, wherein the distal tip defines a straightener suitable to straighten a curved distal tip of the guide wire.

11. The apparatus of claim 1, further comprising a holder connected to a proximal end of the body and in communication with the first axial passageway, the holder storing a portion of the wire guide for delivery into the vessel

12. The apparatus of claim 1, further comprising a lock selectively engaging the advancement wheel.

13. The apparatus of claim 12, wherein the first advancement wheel is radially adjustable, and wherein the lock selectively and independently fixes both the rotation and the radial position of the first advancement wheel.

14. The apparatus of claim 1, further comprising a second advancement wheel rotatably attached to the body and positioned adjacent the first axial passageway, the first and second advancement wheels positioned relative to each other to define the grasping area therebetween.

15. A wire guide apparatus for introducing a wire guide into a vessel through a medical device, the apparatus comprising:
a body defining a first axial passageway therein;
a straightener attached to a distal end of the body, the straightener defining a second axial passageway in communication with the first axial passageway, the straightener having a tapered end portion;
a first advancement wheel rotatably attached to the body and positioned adjacent the first axial passageway, the first advancement wheel defining a grasping area contacting the wire guide and axially translating the wire guide relative to the body;
a valve sealingly engaging the wire guide, the valve located distally relative to the first advancement wheel; and
a port defining a third passageway in communication with the second passageway, the port located between a free end of the straightener and the valve.

16. The apparatus of claim 15, wherein the port is unitarily formed with the straightener.

17. The apparatus of claim 15, wherein the straightener is unitarily formed with the body.

18. The apparatus of claim 15, further comprising a lock selectively engaging the advancement wheel.

19. The apparatus of claim 18, wherein the first advancement wheel is radially adjustable, and wherein the lock selectively and independently fixes both the rotation and the radial position of the first advancement wheel.

20. A wire guide kit comprising:
a wire guide apparatus for introducing a wire guide into a vessel through a medical device, the apparatus comprising:
a body defining a first axial passageway therein;
a straightener attached to a distal end of the body, the straightener defining a second axial passageway in communication with the first axial passageway, the straightener having a tapered free end portion;
a first advancement wheel rotatably attached to the body and positioned adjacent the first axial passageway, the wheel defining a grasping area contacting the wire guide and axially translating the wire guide relative to the body;
a holder connected to a proximal end of the body and in communication with the first axial passageway, the holder storing a portion of the guide wire for delivery into the vessel; a wire guide positioned within first and second passageways and within the holder, the wire guide having a curved distal end positioned outside the wire guide apparatus; and
a sealed package containing the wire guide apparatus and the wire guide.
